(19) United States
(12) Patent Application Publication
(10) Pub. No.: US 2018/0071261 A1
(43) Pub. Date: Mar. 15, 2018

(54) CYCLOCREATINE MICROSUSPENSION

(71) Applicant: Lumos Pharma, Inc., Austin, TX (US)

(72) Inventors: Hong Dixon, San Anotnio, TX (US); Chris J. Bemben, San Anotnio, TX (US); Albert M. Zwiener, Helotes, TX (US)

(21) Appl. No.: 15/554,047

(22) PCT Filed: Mar. 9, 2016
(86) PCT No.: PCT/US16/21543

§ 371 Date: Aug. 28, 2017

Related U.S. Application Data
(60) Provisional application No. 62/130,683, filed on Mar. 10, 2015.

(51) Int. Cl. A61K 31/4172; A61K 9/16; A61K 47/02

(52) U.S. Cl. CPC A61K 31/4172; A61K 47/02; A61K 9/1682

(57) ABSTRACT

Provided is a microsuspension comprising cyclocreatine, or an analog or pharmaceutically acceptable salt thereof

### BRIEF DESCRIPTION OF THE FIGURES

FIG. 1 is a schematic showing bead milling apparatus use to prepared cyclocreatine microsuspension

FIG. 2 shows a picture of cyclocreatine microsuspension

FIG. 3 is a micrograph of particles from microsuspension (pre-milling and post-milling).

FIG. 4 shows an example of the representative particle size distribution for microsuspension before milling (A) and after milling (B).

FIG. 5 is a schematic showing a pin mill setup diagram for dry milling useful for the preparation of cyclocreatine microsuspension.

FIG. 6 shows the particle size of cyclocreatine prior to dry milling.

FIG. 7 shows the particle size of a cyclocreatine sample after dry milling for 45 minutes.

### DETAILED DESCRIPTION OF THE INVENTION

It is to be understood that the figures and descriptions of the present invention have been simplified to illustrate elements that are relevant for a clear understanding of the present invention, while eliminating, for the purpose of clarity, many other elements found in typical pharmaceutical compositions and methods of stabilization. Those of ordinary skill in the art will recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art. Furthermore, the embodiments identified and illustrated herein are for exemplary purposes only, and are not meant to be exclusive or limited in their description of the present invention.

The present invention generally relates to the surprising discovery of an aqueous pharmaceutical composition suitable for providing cyclocreatine, or analogs thereof, with sufficient bioavailability to allow for oral administration. The pharmaceutical composition is a microsuspension comprising particles of cyclocreatine, or analogs thereof, dispersed in an aqueous medium. Cyclocreatine, or analogs or pharmaceutically acceptable salts thereof, can, thus, be provided in an aqueous microsuspension with sufficient solubility, dissolution rate, and/or bioavailability to allow for oral administration.

As used herein, the term “cyclocreatine, or analogs thereof” shall mean and include all varieties or forms of cyclocreatine and analogs thereof. Unless otherwise specified, examples of such forms include all pharmaceutically acceptable salts, zwitterions, esters, isomers, stereo isomers, crystalline and amorphous forms. The amount of cyclocreatine in the formulations of the present invention can vary depending on the total overall volume of the formulation and the concentration of the other components. In one embodiment, cyclocreatine or analogs thereof useful in the invention include compounds of formula (I):

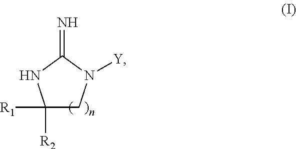

wherein:
Y is CH₂CO₂H, CH₂CONR₁R₂ or CH₂CO₂R₁;
R₁, R₂, independently of each other, is hydrogen, lower alkyl, C₇-C₁₂ alkyl or lower cycloalkyl;

and

n is 1, 2, 3, 4 or 5.

In another embodiment, cyclocreatine or analogs thereof useful in the invention can include compounds of formula (Ia):

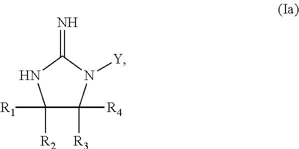

wherein:
Y is CH₂CO₂H, CH₂CONR₁R₂ or CH₂CO₂R₁;
R₁, R₂, R₃, R₄, independently of each other, is hydrogen, lower alkyl, C₇-C₁₂ alkyl or cycloalkyl,
or a pharmaceutically acceptable salt thereof.

A “patient” is a mammal, e.g., a human, mouse, rat, guinea pig, dog, cat, horse, cow, pig, or non-human primate, such as a monkey, chimpanzee, baboon or rhesus monkey, and the terms “patient” and “subject” are used interchangeably herein.

Representative “pharmaceutically acceptable salts” include, e.g., water-soluble and water-insoluble salts, such as the acetate, amsonate (4,4-diaminostilbene-2, 2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. Additional pharmaceutically acceptable salt forms at the carboxylate function would include lithium, sodium, and potassium.

A “therapeutically effective amount” when used in connection with cyclocreatine is an amount effective for treating or preventing a cyclocreatine-regulated disease or disorder.

The microsuspension comprises micronized particles of cyclocreatine, or analogs or pharmaceutically acceptable salts thereof. The micronized particles have a particle diameter, as characterized by a D₉₀ value, in the range of from 1 to 50 microns, in another embodiment from 1 to 30 microns, in a further embodiment from 1 to 20 microns and in a still further embodiment from 1 to 10 microns. Particle size analysis to determine D₉₀ values can be conducted by various techniques know in the art, such as, for example, techniques based on light scattering and image analysis. The concentration of particles of cyclocreatine, or analogs thereof, of the microsuspension can be in the range of from 0.1 to 500 mg/mL, in another embodiment in the range of from 50-150 mg/mL, in a further embodiment of 1 to 40 mg/mL, and in another embodiment in the range of from 2 to 30 mg/mL. Examples include microsuspensions having concentrations of 2 mg/mL, 5 mg/mL, 10 mg/mL, and 20 mg/mL.

In one embodiment, the microsuspension is in an aqueous medium comprising water and optionally other water miscible solvents. Typically, the aqueous medium comprises in the range of from 99.99% to 50%, in another embodiment 95% to 85%, water based on the weight of the aqueous medium.

The microsuspension optionally comprises a stabilizer. The stabilizer is dissolved in the aqueous medium used for the preparation of the microsuspension of cyclocreatine, or analogs thereof. Examples of suitable stabilizers include cellulose ether polymers, such as, hydroxy propyl methyl cellulose (HPMC), methyl cellulose (MC), and hydroxy propyl cellulose (HPC). Suitable amounts of the stabilizer in the microsuspension include 0.01% to 10% w/v, in another embodiment 0.05% to 5% w/v.

The microsuspension can optionally comprise a surfactant. Suitable surfactants include cationic, anionic, and nonionic surfactants. One surfactant or suitable mixture of surfactants may be employed in the microsuspension. Specific examples of suitable surfactants include, but are not limited to, sorbitan esters such as polyoxyethylene (20) sorbitan monooleate, sodium alkyl sulfates such as sodium lauryl sulfate, and/or polyoxyethylene-polyoxypropylene-polyoxyethylene triblock copolymers such as PLURONIC® surfactants (ICI Americas, Delaware). The microsuspension may comprise from 0.01 to 10%, in another embodiment 0.01 to 2%, w/v surfactant based on the volume of the microsuspension.

The microsuspension can optionally comprise a suspending agent to minimize or prevent agglomeration and/or precipitation of the particles of cyclocreatine, or analogs thereof. Suitable suspending agents include alginate, gelatin, carbomers, various gums (e.g., carragenan acacia) and microcrystalline cellulose such as, for example, AVICEL® PH 101, PH 103, PH 105, and PH 200 microcrystalline cellulose (FMC Corporation, Delaware). One or more suspending agents may be employed in the microsuspension. The microsuspension may comprise an amount of suspending agent in the range of from 0.1 to 10% w/v, in another embodiment from 0.5 to 5% w/v, based on the volume of the microsuspension.

The microsuspension can optionally comprise other additives and/or formulation adjuvants. Examples includes flavoring agents and sweeteners such as sorbitol, mannitol, aspartame, sucrose, and other commercially available sweeteners. One sweetener is Simple Syrup, a solution of sucrose in water used in pharmaceutical formulations. Other additives include, buffers such as pharmaceutically acceptable weak acids, weak bases, or mixtures thereof. Preferred buffers are water soluble materials such as phosphoric acid, acetic acid, their salts, or mixtures thereof, which can be use maintain a pH in the range of 5-7 in the microsuspension. Also, preservatives may be added, such as methyl or propyl parabens, or mixtures thereof.

### Preparation of Formulations of the Invention

Cyclocreatine or analogs thereof can be manufactured by any known process in the art. In one embodiment, cyclocreatine can be made using cyanamide as shown in Scheme 1 below:

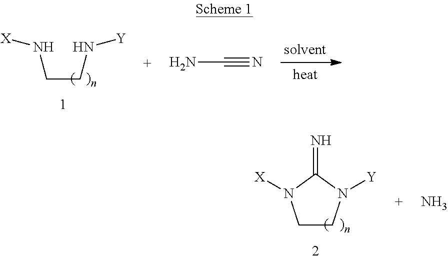

In detail, Scheme I shows a method for the preparation of various cyclic analogs of creatine (2) by the condensation of diamines or their salts (1) with cyanamide in a suitable solvent. In one embodiment, 1 (X=H, Y=CH₂CO₂H, n=1) is reacted with cyanamide in ethanol or water at 25-100° C. to afford 2 (X=H, Y=CH₂CO₂H, n=1). The diamine may be a purified substance or a mixture containing approximately 20-99% 6. The product 2 may, in some embodiments, be further purified by crystallization or slurry from water or another suitable solvent.

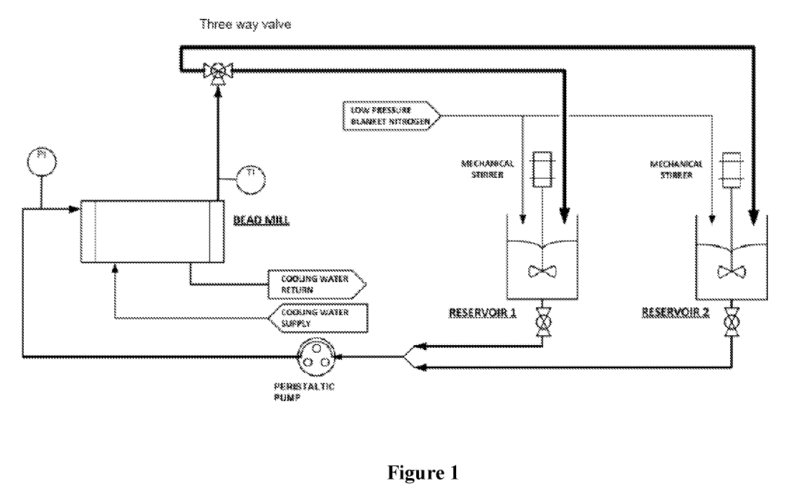

Microsuspension of the invention comprising cyclocreatine, or analogs thereof, can be prepared using any device or method commonly used in the art. In one embodiment, a Glen Dyno Mill can be used with grinding media such as zirconia, glass, ceramics, special polymers or combinations thereof, to create shearing and impacting forces to develop microsuspension formulations with solid concentrations of cyclocreatine in water at approximately 200 mg/mL. The grinding media can range in size from 1.0 to 1.5 mm. A schematic of a wet milling apparatus useful in the invention is shown in FIG. 1. Parameters in this process include, for example, grinding media size, viscosity of suspension medium, solid concentration in the suspension medium, rotor speed and grinding time.

### Methods of Use

The formulations of the invention can be used for the treatment of, for example, a cognitive dysfunction in a subject by modulating, e.g. increasing, brain energy metabolism. Brain energy metabolism can be modulated by administering to the subject an effective amount of a brain energy metabolism modulating compound. In a further embodiment, the subject's brain energy metabolism is normal, after the administration of the brain energy modulating compound.

The term “brain energy metabolism” includes aerobic metabolism, anaerobic metabolism, glycolytic metabolism, mitochondrial metabolism, and the generation of energy buffers such as adenylate kinase and creatine kinase, which generate energy in the brain. It also includes energy metabolism in the subject's neural or glial cells. Brain energy metabolism can be increased by increasing the ATP or creatine phosphate concentration, or by decreasing the concentration of ADP, GDP, AMP, or other mono- or di-phosphorylated nucleotides. Brain metabolism can be increased by the administration of brain energy modulating compounds.

The term “cognitive dysfunction” includes learning dysfunction, autism, attention deficit disorders, fragile X syndrome, obsessive-compulsive disorders, speech dysfunction, speech deficits, learning disabilities, impaired communication skills, mental retardation, low IQ, short term memory dysfunction, spatial learning dysfunction, and inborn errors of metabolism affecting the brain (such as, but not limited to creatine transporter dysfunction, GAMT, and AGAT). Cognitive dysfunction also includes states of altered cognitive, expressive and behavioral function. In an embodiment, GAMT deficiency is not a cognitive dysfunction of the invention. In one embodiment, the term “cognitive dysfunction” does not include neurodegenerative disorders.

The term “creatine transporter dysfunction” includes a disorder characterized by an inborn error creatine synthesis or of the creatine transporter or other aberrant creatine transport function in the brain. The aberrant creatine transport function in the brain may cause the subject to suffer from a low concentration of creatine in the brain of a subject suffering from creatine transporter dysfunction. In this disorder, impaired energy metabolism is believed to be associated with impaired learning dysfunction and cognitive function. It was found that treatments of similar neurological or cognitive dysfunctions do not tend to target improving metabolism and/or energy metabolism of the brain, neural cells, or glial cells. The invention also pertains, at least in part, to methods of treating subject with a creatine transport deficiency in the brain.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### Preparation of Microsuspension Formulation

To a milling vessel size of 0.6 liter of a Glen Dyno Mill about 70% volume of zirconia grinding media was charged. An agitator shaft activated the media, creating shearing and impacting forces. The rotation of the agitator imparted energy to the surrounding media and fractures the cyclocreatine solids suspended in water, resulting in overall reduction in particle size.

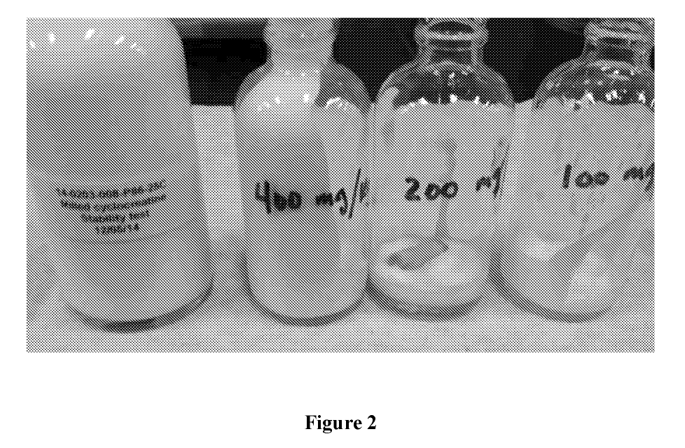

Water was charged into a stainless steel vessel equipped with overhead agitator and blanketed with nitrogen followed by the addition of cyclocreatine. The mixture was pumped via a peristaltic pump into the grinding chamber. The milled sample exiting the chamber was collected in another stainless steel vessel (also equipped with an overhead agitator and blanked with nitrogen) until the first vessel was empty at which time a three way valve was switched as to continue pumping the mixture in one of the vessels into the grinding chamber. The drug concentration in the suspension ranged from 10-20% w/v. The milling chamber had a rotor fitted with disks that were accelerated with speed up to 3344 rpm. The rotation of the disk accelerated the milling media radially. The suspension mixture flowed axially through the milling chamber where the shear forces generated during impaction of the milling media with the solid particles provided the energy input to fracture the drug into nanometer-sized particles. Up to 40% nanoparticles were observed. In another embodiment, up to about 10% nanoparticles by volume were observed. The temperature inside the grinding chamber was controlled by circulating coolant through the outer jacket. The resultant microsuspension had good flow characteristics and appeared milky (FIG. 2).

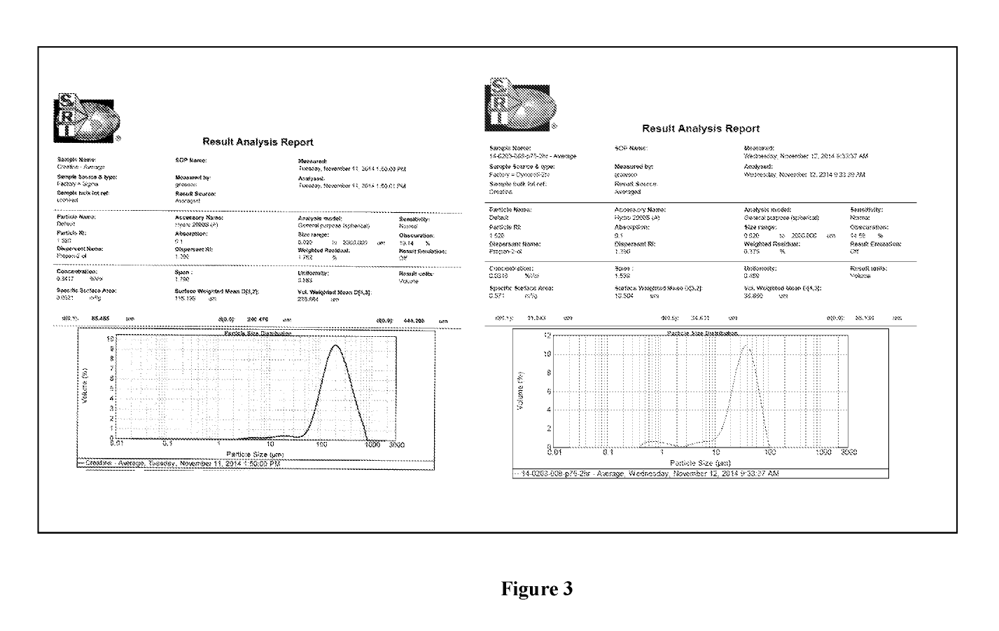

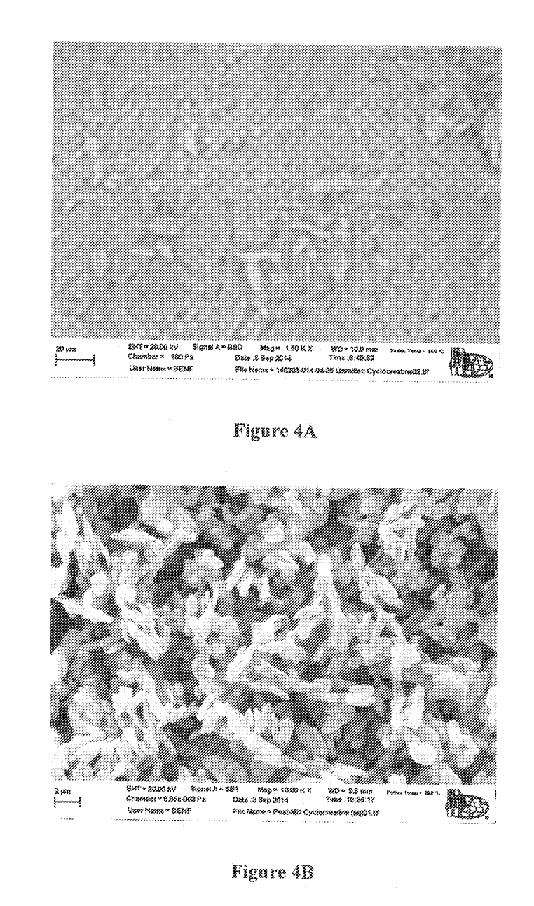

With a given suspension composition and given energy (grinding bead size, rotor speed), the process was found to be quite robust. For example a 200 mg/mL solid concentration of formulation processed in a 0.6 liter sized grinding chamber filled with 1.0 mm diameter zirconia beads and a 3344 rpm agitator speed for a total of two hours produced samples with similar final particle size in three repeated experiments. In all instances, the particle size decreased markedly within 30 min processing time from the initial 400 μm to about 80 μm, but with little change observed between 30 and 120 min. Further prolonging in the milling times was not beneficial, because decreased particle sizes were not achieved. In addition to particle size analysis (FIG. 3), the SEM micrographs (FIG. 4) further confirmed that the milling process as effective in converting the original cyclocreatine particles into the low micron range.

### Example 2

### Drug Stability

The cyclocreatine samples were analyzed before and after milling (Example 1) to assess the effect of milling on the drug stability. As demonstrated in Table 1 below, the milling process of Example 1 caused negligible drug degradation:

TABLE 1
Cyclocreatine particle size and purity after milling process

| | Particle Size (μm, | Cyclocreatine |
|---|---|---|
| | Volume weighted | degradation after milling |
| Process condition | average) | process (%) |
| Un-milled cyclocreatine | 400 | 0 |
| Wet bead mill | 1-10 | 0 |

### Example 3

### Dilution Injection Test and Stability Test

Approximately 2.5 mL suspension formulation of cyclocreatine in water (˜200 mg/mL) was loaded into each of the 10 mL syringe equipped with gavage tubing. The syringe and plunger assembly with the gavage tube was compressed and the resulting sample analyzed (Table 2). This study confirmed cyclocreatine water suspension formulation is suitable for use in animal studies could be easily and accurately delivered at ambient temperatures.

TABLE 2
Concentration of Cyclocreatine in the
suspension formulation over 40-days

| Time | Cyclocreatine concentration in |
|---|---|
| (day) | the suspension sample (mg/g) |
| 0 | 177.1 ± 0.5 |
| 7 | 174.6 ± 1.0 |
| 11 | 175.7 ± 1.0 |
| 26 | 176.9 ± 0.5 |
| 40 | 180.1 ± 1.5 |

A more concentrated cyclocreatine suspension sample was prepared after removing deionized water from the milled sample. The concentration of cyclocreatine in the resulting sample was found to be about 360.2±4.1 mg/g, and it maintained shear-thinning property that this more concentrated suspension was easily injected through a 22 gauge syringe needle.

A preliminary rat oral-gavage dosing test with cyclocreatine suspension sample (14-0203-008-p39-1) showed uniform dose delivery (Table 3).

TABLE 3
Cyclocreatine dose delivery (2.5 mL suspension)

| Amount of cyclocreatine | Average amount of cyclocreatine |
|---|---|
| delivered in 2.5 mL sample | delivered in 2.5 mL sample through a |
| through a syringe (mg) | syringe (mg) |
| 422.6 | 422.7 |
| 410.0 | |
| 435.6 | |

As shown in the Examples above, suspension formulations of cyclocreatine solid microparticles in water showed excellent chemical stability and good properties for oral dosing.

### Example 4

### Dry Milling

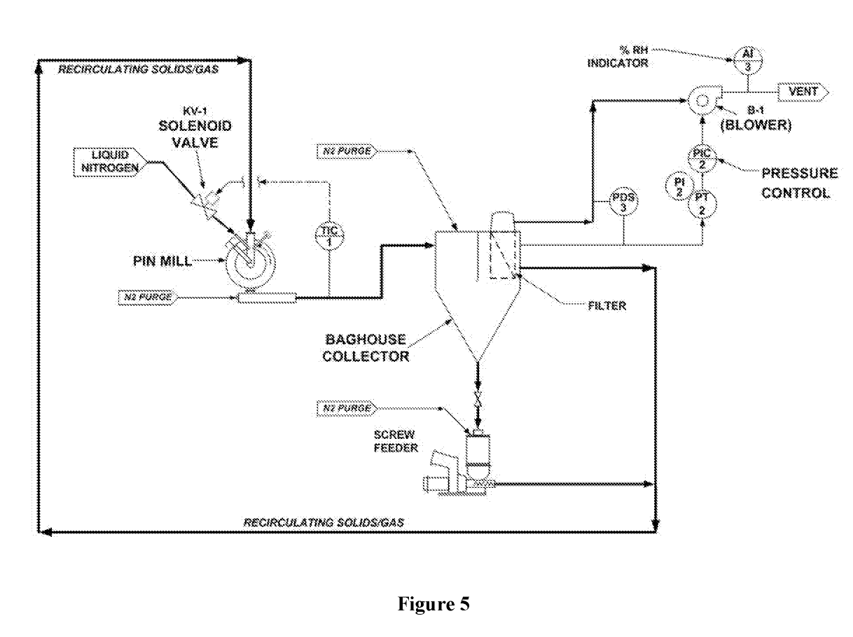

A dry milling process was conducted on cyclocreatine using a centrifugal impact mill (typically referred to in the art as a pin mill). As shown in FIG. 5, a Munsen CIM-18 pin mill was arranged in a powder/nitrogen recirculation loop batch milling without stop/start cycles. Nitrogen gas purges were installed in three places: 1) pin mill outlet, 2) top of the baghouse collector, and 3) screw feeder hopper. Relative humidity indicator AI-2 was used to indicate the efficacy of the nitrogen purge.

About 2-kg of cyclocreatine (API) was introduced into the mill. Operation of the pin mill caused recirculation of nitrogen gas and pneumatic conveying of the powder from the screw feeder discharge. Liquid nitrogen was used to cool the pin mill discharge through closed loop control via temperature controller TIC-2 and solenoid valve KV-2 (pulse width modulation). Liquid nitrogen pulses flowed through a pressure spray nozzle inserted within the 3 inch diameter pin mill powder feed port. Powder/nitrogen discharge from the pin mill was pneumatically conveyed to the baghouse collector, which was vibrated continuously (pneumatic vibrator) to facilitate powder return to the screw feeder.

Blower B-1 was used to maintain vacuum pressure on the collector and contain powder while minimizing nitrogen flow through the filter. Minimizing nitrogen flow through the filter maintained the highest filter efficiency possible in this system. Further filter efficiency was achieved by inhibiting filter pulse operation through pressure differential switch PDS-1 until 2 inches of water column pressure drop was achieved. At the end of the run, pulse inhibition was overridden to facilitate collection.

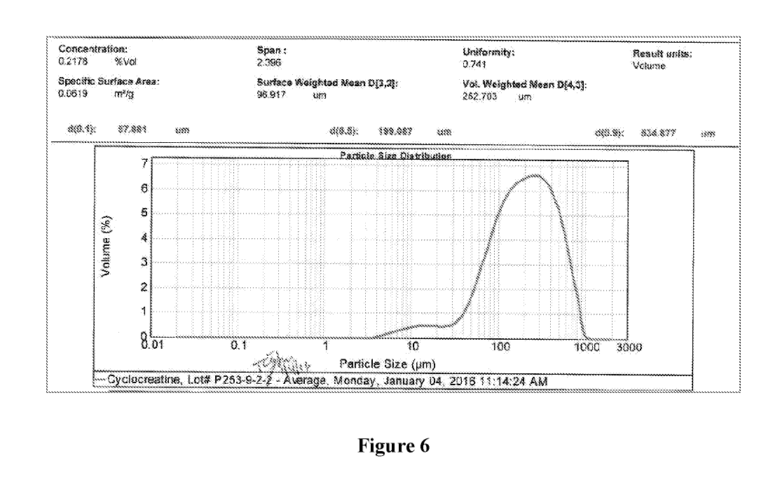

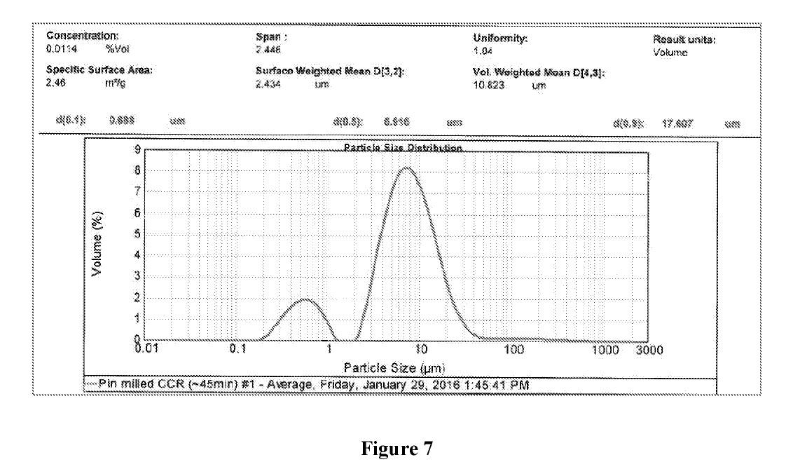

After milling for about 45 min, the particle size of the micronized API appeared to have been reduced uniformly for the entire batch of sample. The particle size data of the API before and after milling is shown in FIGS. 6 and 7. The API was found to be stable during the milling process.

The collected micronized API was then formulated with water to form an aqueous microsuspension. 20.0 grams of cyclocreatine was weighed into a graduated vessel. Approximately 80 mL of water was added and the solution mixed to allow partial dissolution. After mixing for 5 minutes, water was added to dilute to a final volume of 100 mL. The resulting suspension had a nominal strength of 200 mg cyclocreatine per mL of suspension. The solubility of cyclocreatine in water was 17 mg/mL.

The invention is further described in the following numbered paragraphs:

1. A pharmaceutical oral dosage form, comprising an aqueous microsuspension comprising cyclocreatine, or an analog or pharmaceutically acceptable salt thereof.
2. The pharmaceutical oral dosage form according to paragraph 1, wherein said cyclocreatine, or analog or pharmaceutically acceptable salt thereof, has a volume weighted average particle size of 0.1 to 500 μm.
3. The pharmaceutical oral dosage form according to paragraph 1, wherein said cyclocreatine, or analog or pharmaceutically acceptable salt thereof, has a volume weighted average particle size of 0.1 to 10 μm.
4. An aqueous microsuspension comprising cyclocreatine, or an analog or pharmaceutically acceptable salt thereof.
5. The aqueous microsuspension according to paragraph 4, wherein said cyclocreatine, or analog or pharmaceutically acceptable salt thereof, has a volume weighted average particle size of 0.1 to 500 μm.
6. The aqueous microsuspension according to paragraph 4, wherein said cyclocreatine, or analog or pharmaceutically acceptable salt thereof, has a volume weighted average particle size of 0.1 to 10 μm.
7. An aqueous microsuspension comprising cyclocreatine, or an analog or pharmaceutically acceptable salt thereof, prepared by a process comprising the steps of:
charging a milling vessel with grinding media and water;
pumping cyclocreatine or an analog or pharmaceutically acceptable salt thereof into said milling vessel; and
fracturing said cyclocreatine, or an analog or pharmaceutically acceptable salt thereof to form said aqueous microsuspension.
8. A method of making an aqueous microsuspension comprising cyclocreatine, or an analog or pharmaceutically acceptable salt thereof, comprising the steps of:
charging a milling vessel with grinding media and water;
pumping cyclocreatine or an analog or pharmaceutically acceptable salt thereof into said milling vessel; and
fracturing said cyclocreatine, or an analog or pharmaceutically acceptable salt thereof to form an aqueous microsuspension.
9. The method according to paragraph 8, further comprising the step of blanketing the milling vessel with nitrogen.
10. The method according to paragraph 8, wherein said cyclocreatine, or an analog or pharmaceutically acceptable salt thereof, is at a concentration of 1 to 50% w/v.
11. The method according to paragraph 8, wherein said cyclocreatine, or an analog or pharmaceutically acceptable salt thereof, is at a concentration of 10 to 20% w/v.
12. A method of making micronized cyclocreatine, or an analog or pharmaceutically acceptable salt thereof, comprising the steps of:
placing cyclocreatine or an analog or pharmaceutically acceptable salt thereof into a centrifugal impact mill vessel;
activating said centrifugal impact mill; and
fracturing said cyclocreatine, or an analog or pharmaceutically acceptable salt thereof to form micronized cyclocreatine.
13. An aqueous microsuspension comprising cyclocreatine, or an analog or pharmaceutically acceptable salt thereof, prepared by a process comprising the steps of:
placing cyclocreatine or an analog or pharmaceutically acceptable salt thereof into a centrifugal impact mill vessel;
activating said centrifugal impact mill;
fracturing said cyclocreatine, or an analog or pharmaceutically acceptable salt thereof to form micronized cyclocreatine;
collecting said micronized cyclocreatine or an analog or pharmaceutically acceptable salt thereof from said centrifugal impact mill vessel; and
formulating said collected micronized cyclocreatine or an analog or pharmaceutically acceptable salt thereof into an aqueous formulation.
14. A method for treating creatine transporter dysfunction, comprising the step of administering a therapeutically effective amount of the pharmaceutical oral dosage form of paragraph 1 to a subject in need thereof.

It is to be understood that the invention is not limited to the particular embodiments of the invention described above, as variations of the particular embodiments may be made and still fall within the scope of the appended claims.

1. A pharmaceutical oral dosage form, comprising an aqueous microsuspension comprising cyclocreatine, or an analog or pharmaceutically acceptable salt thereof.

2. The pharmaceutical oral dosage form according to claim 1, wherein said cyclocreatine, or analog or pharmaceutically acceptable salt thereof, has a volume weighted average particle size of 0.1 to 500 μm.

3. The pharmaceutical oral dosage form according to claim 1, wherein said cyclocreatine, or analog or pharmaceutically acceptable salt thereof, has a volume weighted average particle size of 0.1 to 10 μm.

4. An aqueous microsuspension comprising cyclocreatine, or an analog or pharmaceutically acceptable salt thereof.

5. The aqueous microsuspension according to claim 4, wherein said cyclocreatine, or analog or pharmaceutically acceptable salt thereof, has a volume weighted average particle size of 0.1 to 500 μm.

6. The aqueous microsuspension according to claim 4, wherein said cyclocreatine, or analog or pharmaceutically acceptable salt thereof, has a volume weighted average particle size of 0.1 to 10 μm.

7. An aqueous microsuspension comprising cyclocreatine, or an analog or pharmaceutically acceptable salt thereof, prepared by a process comprising the steps of:
charging a milling vessel with grinding media and water;
pumping cyclocreatine or an analog or pharmaceutically acceptable salt thereof into said milling vessel; and
fracturing said cyclocreatine, or an analog or pharmaceutically acceptable salt thereof to form said aqueous microsuspension.

8. A method of making an aqueous microsuspension comprising cyclocreatine, or an analog or pharmaceutically acceptable salt thereof, comprising the steps of:
charging a milling vessel with grinding media and water;
pumping cyclocreatine or an analog or pharmaceutically acceptable salt thereof into said milling vessel; and
fracturing said cyclocreatine, or an analog or pharmaceutically acceptable salt thereof to form an aqueous microsuspension.

9. The method according to claim 8, further comprising the step of blanketing the milling vessel with nitrogen.

10. The method according to claim 8, wherein said cyclocreatine, or an analog or pharmaceutically acceptable salt thereof, is at a concentration of 1 to 50% w/v.

11. The method according to claim 8, wherein said cyclocreatine, or an analog or pharmaceutically acceptable salt thereof, is at a concentration of 10 to 20% w/v.

12. A method of making micronized cyclocreatine, or an analog or pharmaceutically acceptable salt thereof, comprising the steps of:
placing cyclocreatine or an analog or pharmaceutically acceptable salt thereof into a centrifugal impact mill vessel;
activating said centrifugal impact mill; and
fracturing said cyclocreatine, or an analog or pharmaceutically acceptable salt thereof to form micronized cyclocreatine.

13. An aqueous microsuspension comprising cyclocreatine, or an analog or pharmaceutically acceptable salt thereof, prepared by a process comprising the steps of:
placing cyclocreatine or an analog or pharmaceutically acceptable salt thereof into a centrifugal impact mill vessel;
activating said centrifugal impact mill;
fracturing said cyclocreatine, or an analog or pharmaceutically acceptable salt thereof to form micronized cyclocreatine;
collecting said micronized cyclocreatine or an analog or pharmaceutically acceptable salt thereof from said centrifugal impact mill vessel; and
formulating said collected micronized cyclocreatine or an analog or pharmaceutically acceptable salt thereof into an aqueous formulation.

14. A method for treating creatine transporter dysfunction, comprising the step of administering a therapeutically effective amount of the pharmaceutical oral dosage form of claim 1 to a subject in need thereof.
